(19) United States
(12) Patent Application Publication
(10) Pub. No.: US 2026/0002085 A1
(43) Pub. Date: Jan. 1, 2026

(54) SYSTEMS AND PROCESSES FOR THE PRODUCTION OF HYDROCARBON PRODUCTS FROM CRUDE AND HEAVY HYDROCARBON FEEDSTOCKS

(71) Applicant: SABIC GLOBAL TECHNOLOGIES B.V., BERGEN OP ZOOM (NL)

(72) Inventors: Ravichander NARAYANASWAMY, Bangalore (IN); Prashil Prakash LAKHETE, Bangalore (IN); Kenneth Francis LAWSON, Bangalore (IN); Alexander STANISLAUS, Bangalore (IN); Abdulrahman Shahid SARANG, Bangalore (IN)

(73) Assignee: SABIC GLOBAL TECHNOLOGIES B.V., BERGEN OP ZOOM (NL)

(21) Appl. No.: 18/992,712

(22) PCT Filed: Jul. 6, 2023
(86) PCT No.: PCT/EP2023/068774

§ 371 Date: Jan. 9, 2025

(30) Foreign Application Priority Data
Jul. 9, 2022 (IN) 202241039478

(51) Int. Cl. C10G 69/06; C07C 4/04

(52) U.S. Cl. CPC C10G 69/06; C07C 4/04; C10G 2300/1037; C10G 2400/04; C10G 2400/06

(57) ABSTRACT

A system and method for production of olefin products, diesel and fuel oil from a hydrocarbon feedstock is disclosed. The system includes feeding a bottom stream of flashed crude oil to an aquaprocessing unit, aquaprocessing the flashed crude to yield a gas stream that is sent to a steam cracker, diesel and fuel oil, and a liquids stream that is send to the steam cracker to produce olefins. Further processes yield ethylene, propylene and butylene. Pitch and/or coke may also be produced.

### CROSS REFERENCE TO RELATED APPLICATIONS

None.

### BACKGROUND OF THE INVENTION

### A. Field of the Invention

The invention generally concerns systems and methods for producing diesel and low sulfur fuel oil or lube oil products from a hydrocarbon feedstock while also producing high value chemical products. A system can include an aquaprocessing unit fluidly coupled to a crude separation unit (a flash vessel, a distillation column or a stripping unit) such that heavy flashed crude from the separation unit is fed to the aquaprocessing unit for processing under conditions to yield diesel, very low sulfur fuel oil and/or pitch, as well as a liquid fraction having a boiling point in the range of 35 to 400° C., and a gas fraction. A steam cracker is also provided and the liquid fraction from the aquaprocessor is fed to the steam cracker, along with the gas fraction from the aquaprocessor and optionally the gas fraction from the crude flash unit, and steam cracked under conditions to produce olefins. Additionally in certain configurations, the hydrocarbon feedstock is first processed in a fluid catalytic cracking unit to produce high value chemicals and liquids; the liquids from FCC unit are fed to aquaprocessing unit and then converted further to olefins in steam cracker. The first step of FCC helps in reducing coke lay down in aquaprocessing unit during high conversion and also helps in altering the P/E ratio to higher values. Refer aquaprocessing applications for write up.

Aquaprocessing is the hydrocracking of hydrocarbons in the presence of (a) one or more aquaprocessing catalysts and (b) a solvent to keep asphaltenes dissolved, at a temperature in the range of 280 to 550° C. and a pressure in the range of 40 to 200 barg. In embodiments of the invention, the aquaprocessing conditions can include a hydrogen to hydrocarbon ratio from 200 to 2000 NL/L of liquid feed and the aquaprocessing can be carried out with or without added steam. The aquaprocessing catalyst may include a dissolved portion and a dispersed portion. The dissolved portion of the catalyst includes an organometallic compound having one or more of Ni, Mo, Co, W, Zr. The dissolved portion of the catalyst may include metal naphthenates and/or octanoates having hydrogenation activity. The dispersed portion of the catalyst is a selection from the list consisting of: an alkali metal hydroxide or oxide, Ni—Mo oxides or sulphides, Co—Mo oxides or sulphides, W—Mo oxides or sulphides on alumina or zeolites or any combination of these having hydro-processing and/or hydrogen transfer activity.

### B. Description of Related Art

Olefins (e.g., ethylene), are basic building blocks for a variety of commercially valuable polymers. Naturally occurring sources of olefins do not exist in commercial quantities. Therefore, polymer producers rely on methods for converting the more abundant lower alkanes into olefins. The method of choice for today's commercial scale producers is steam cracking, a highly endothermic process where steam-diluted alkanes are subjected briefly to a temperature of at least 800° C. The fuel demand to produce the required temperatures and the need for equipment that can withstand that temperature add significantly to the overall cost. Also, the high temperature promotes the formation of coke which accumulates within the system, resulting in the need for costly periodic reactor shut-down for maintenance and coke removal.

Processes and systems to produce olefins from crude oil have been described. For example International Patent Application No. WO 2017/133975 to Ward et al., describes an integrated process to convert crude oil into petrochemical products that includes crude oil distillation, hydrocracking and steam cracking to produce petroleum products. Unfortunately, high conversion to ethylene in steam crackers leads to more loss of gases as methane and this leads to lower carbon efficiency. Also, when attempting to convert straight run crude cuts and all streams are fed directly from a hydrocracker to a steam cracker, the capacity of the steam cracker goes up, not only because the full flow is directed to the steam cracker, but also because recycle of pyoil streams to the steam cracker increase as heavier feeds get converted in the steam cracker

Overall, while the technologies of producing olefins exist, they can be energy and material inefficient and expensive.

Diesel fuel is an important fuel for the automotive and trucking industries. Fuel oil is an important heating fuel for commercial and residential use, and should also be low in sulfur content.

Typically, these products have been prepared by subjecting various heavier crude oil cuts, such as vacuum residue, to catalytic hydrocracking processes. These hydrocracking processes are energy intensive and do not always provide sufficient yields, and equipment costs are high.

Thus, while technologies for producing olefins, diesel and fuel oil exist, new and more efficient systems and processes for preparing these products are desired. This invention allows for withdrawing diesel as a product when required.

### SUMMARY OF THE INVENTION

A discovery has been made that provides a solution to at least one of the problems associated with production of diesel and/or fuel oil. An aspect relates to a method for producing olefins and diesel and/or fuel oil. The method includes feeding a hydrocarbon feed into a first separation unit to produce a first stream having a boiling point less than 200° C. and a second stream having a boiling point above 200° C.; feeding the second stream to a first aquaprocessing unit having a first catalyst therein and aquaprocessing the third stream at a temperature of from greater than 400° C. to 500° C. to produce an aquaprocessing effluent containing 95 wt. % hydrocarbons having boiling below 350° C.; separating the aquaprocessing effluent to form a first stream comprising the diesel having a boiling range of from 220 to 370° C.; a third stream comprising fuel oil, a liquid stream having a boiling point in the range of from 35 to 400° C., and a first gaseous stream having a boiling point of less than 200° C.; feeding the first gaseous stream and the first stream to a steam cracker. The liquid stream may be fed to the steam cracker. The first stream may be fed to the steam cracker to produce olefins.

An aspect relates to a method for producing olefins and diesel and/or fuel oil, the method including the steps of feeding a hydrocarbon feed into a first separation unit to produce a first stream having a boiling point less than 200° C., and a second stream having a boiling point above 200° C.; feeding the second stream to a first aquaprocessing unit having a first catalyst therein and aquaprocessing the third stream at a temperature of from greater than 400° C. to 500° C. to produce an aquaprocessing effluent containing 95 wt % hydrocarbons having a boiling point below 350° C.; separating the aquaprocessing effluent to form a first stream comprising diesel having a boiling range of from 220 to 370° C.; a third stream comprising fuel oil, a liquid stream having a boiling point in the range of from 35 to 400° C., and a second gaseous stream having a boiling point of less than 200° C.; combining the second gaseous stream and the first stream to form a combined gas stream; feeding the combined gas stream to a steam; and steam cracking the liquid stream in the steam cracker to produce olefins.

An aspect relates to a method for producing olefins and diesel and/or fuel oil, the method includes the steps of feeding a hydrocarbon feed into a first separation unit to produce a first stream having a boiling point less than 200° C., and a second stream having a boiling point above 200° C.; feeding the second stream to a first aquaprocessing unit having a first catalyst therein and aquaprocessing the third stream at a temperature of from greater than 400° C. to 500° C. to produce an aquaprocessing effluent containing 95 wt. % of hydrocarbons having a boiling point below 350° C.; separating the aquaprocessing effluent to form a first stream comprising diesel having a boiling range of from 220 to 370° C.; a third stream comprising fuel oil, a liquid stream having a boiling point in the range of from 35 to 400° C., and a third gaseous stream having a boiling point of less than 200° C.; combining the third gaseous stream and the first stream to form a combined gas stream; feeding the combined gas stream to a second flash unit to produce a flashed product; separating the flashed product into a stream comprising ethane, a stream comprising butane and a stream comprising propane; feeding the steam comprising ethane to a steam cracker; and steam cracking the stream comprising ethane in the steam cracker to produce olefins.

An aspect relates to a method for processing a hydrocarbon, the method including the steps of feeding a hydrocarbon feed into an optional first separation unit to produce a first stream having a boiling point less than 200° C. and a second stream having a boiling point above 200° C.; feeding the first stream to a first steam cracker; feeding the second stream or the hydrocarbon feed to a first fluid catalytic cracker having a first catalyst therein and cracking the second stream in the first fluid catalytic cracker by contacting the second stream with the first catalyst at a temperature of from 450° C. to 730° C. to produce a first liquid effluent having a boiling point ranging from 35 to 550° C. and a gaseous effluent stream having C1 to C4 hydrocarbons; feeding the first liquid stream to a first aquaprocessing unit having a first catalyst containing particulate and dissolved catalysts; therein and aquaprocessing the first liquid stream at a temperature of from 330 to 500° C. to produce an aquaprocessing effluent having a boiling point ranging up to 400° C.; separating the aquaprocessing effluent to form a second liquid stream having a boiling point range of from 200to 400° C., and a gaseous stream having a boiling point of less than 200° C.; feeding the gaseous stream and the second liquid stream to the first steam cracker; and steam cracking the liquid stream in the steam cracker to produce olefins.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The term “C #hydrocarbons”, wherein “#” is a positive integer, is meant to describe all hydrocarbons having #carbon atoms. Moreover, the term “C #+ hydrocarbons” is meant to describe all hydrocarbon molecules having #or more carbon atoms. Accordingly, the term “C2+ hydrocarbons” is meant to describe a mixture of hydrocarbons having 2 or more carbon atoms. The term “C2+ alkanes” accordingly relates to alkanes having 2 or more carbon atoms.

“Cracking” refers to a process involving decomposition and molecular recombination of organic compounds to produce a greater number of molecules than were initially present. In cracking, a series of reactions take place accompanied by a transfer of hydrogen atoms between molecules. For example, naphtha may undergo a thermal cracking reaction to form ethene and hydrogen.

“Hydrocarbons” are generally defined as molecules formed primarily by carbon and hydrogen atoms. Hydrocarbons may also include other elements such as, but not limited to, halogens, metallic elements, nitrogen, oxygen, and/or sulfur. Hydrocarbon fluids may include, entrain, or be entrained in non-hydrocarbon fluids such as hydrogen, nitrogen, carbon monoxide, carbon dioxide, hydrogen sulfide, water, and/or ammonia.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The systems and processes of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the systems and methods of the present invention are their abilities to produce olefin products (e.g., ethylene) in a cost and energy efficient manner by having an ethane steam cracker unit capable of receiving ethane from a mixed feed steam cracker unit and feeding the C2+ products produced by the ethane steam cracker unit to the mixed feed steam cracker unit.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 illustrates an embodiment of a system to produce olefins, diesel and/or fuel oil products from a hydrocarbon feed provided to an aquaprocessing unit.

FIG. 2 illustrates an embodiment of a system to produce diesel, fuel oil and olefin products from a hydrocarbon feed provided to an aquaprocessing unit.

FIG. 3 illustrates configuration with an upstream fluid catalytic cracking (FCC) unit.

While the invention is susceptible to various modifications and alternative forms, specific embodiments thereof are shown by way of example in the drawings. The drawings may not be to scale.

### DETAILED DESCRIPTION OF THE INVENTION

A discovery has been made that provides a solution to at least one of the problems associated with preparing olefins, and diesel and/or fuel oil as well. In one aspect, the mixed hydrocarbon feed can be fed to a mixed hydrocarbon steam cracking unit to produce petroleum products and ethane. The ethane can be provided to an independent ethane cracking unit to produce ethylene and/or a C2+ hydrocarbons stream, which can be recycled to the mixed hydrocarbon steam cracking unit. An advantage of this set-up is that the mixed hydrocarbon steam cracking unit can be operated at optimal conditions for C2+ cracking while the ethane steam cracking unit can be operated at optimal conditions to crack ethane. These and other non-limiting aspects of the present invention are discussed in further detail in the following sections with reference to the Figure.

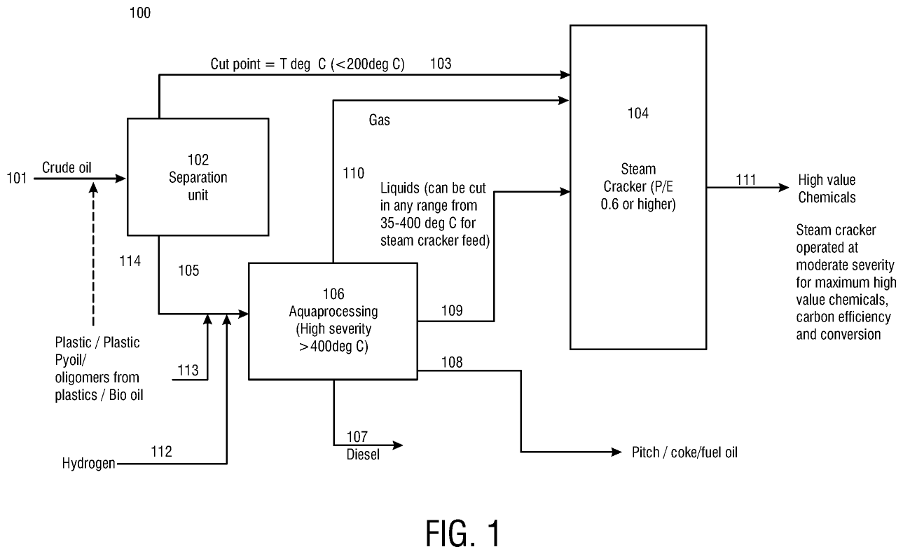

Referring to FIG. 1, system 100 for producing olefin products is described. System 100 can include a crude flash unit 102, a steam cracking unit 104, and aquaprocessing unit 106. Crude oil 101 enters crude flash unit 102 where it is flashed to produce a stream having a cut point below 200° C. 103 and a bottom stream 105 which is fed to aquaprocessing unit 106. Stream 103 may be fed to steam cracker 104. Hydrogen 112 is also fed to aquaprocessing unit 106. The bottom stream 105 is aquaprocessed in the aquaprocessing unit under conditions that produce a liquid stream 109 that is fed to steam cracking unit 104. A gas stream 110 from aquaprocessing unit 106 is also fed to steam cracking unit 106. All feeds in steam cracker 104 may be processed under conditions that produce olefins 111. Aquaprocessing unit will also produce diesel 107 a fuel oil stream 108 containing fuel oil.

Crude oil 101 can be the petroleum extracted from geologic formations in its unrefined form. The term crude oil can also include petroleum that has been subjected to water-oil separations and/or gas-oil separation and/or desalting and/or stabilization. Non-limiting examples of crude oil include Arabian Heavy, Arabian Light, other Gulf crudes, Brent, North Sea crudes, North and West African crudes, Indonesian, Chinese crudes, West Texas crude, and mixtures thereof, but also shale oil, tar sands, gas condensates and bio-based oils. The crude oil used as feed to the process of the present invention preferably is conventional petroleum having an API gravity of more than 20° API as measured by the ASTM D287 standard. In one aspect, the crude oil used in the process of the present invention is a light crude oil having an API gravity of more than 30° API. In another aspect, the crude oil used in the process of the present invention can include Arabian Light Crude Oil. Arabian Light Crude Oil typically has an API gravity of between 32-36° API and a sulfur content of between 1.5-4.5 wt. %.

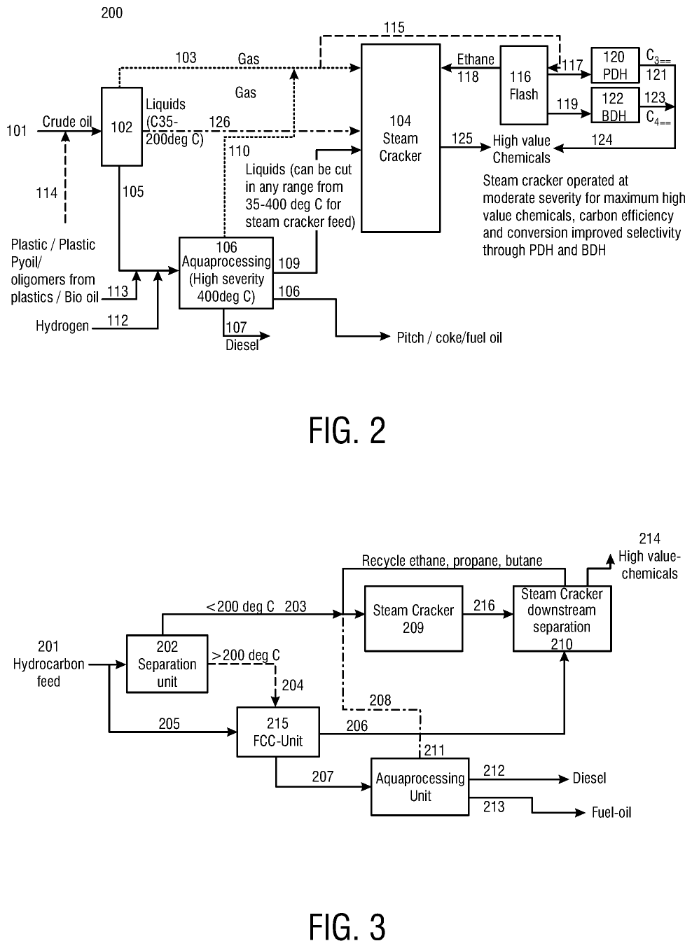

Referring to FIG. 2, an alternative process is shown where in gas stream 110 from aquaprocessing unit 106 is combined with stream 103, and the combined stream is fed to steam cracker unit 104.

Alternatively, the combined gas stream 115 may be fed to a distillation unit 116 wherein ethane 118 is separated and fed to steam cracker 115. A propane stream 117 may also be separated in distillation unit 116 and fed to a propane dehydrogenation unit 120 or propane steam cracker to produce propylene 121. Similarly, a butane stream may be separated from distillation unit 116 and fed to a butane dehydrogenation unit or a butane steam cracker 122 to produce C4 olefins 123 or a butane to ethane conversion unit from which ethane is fed to ethane steam cracker to produce ethylene. Propylene stream 121 and C4 olefin stream 123 may be combined into steam 124 and recovered.

Referring now to FIG. 3, hydrocarbon feed 201, which may be crude oil, is fed to separation unit 202 where it is separated into a stream 203 having a boiling point of less than 200° C., and a stream 204 with a boiling point greater than 200° C.

Stream 203 having a boiling point of less than 200° C. is fed to steam cracker 209 and steam cracked to produce steam cracked effluent 216 which is separated into final products 214 which includes olefins.

Stream 204 with a boiling point greater than 200° C. is fed to an FCC unit 215 which contains an FCC processing catalyst therein, preferably a catalyst containing a zeolite, more preferably ZSM-5, an HY zeolite, an NaY zeolite, a SAPO011 zeolite, anMCM-41 zeolite, and processed under conditions to produce a bottom stream 207 and a top stream 206 which is fed to a downstream separation unit 210. Conditions may include a temperature of from 400 to 900° C., more preferably 500 to 800° C., and more preferably 600 t 700° C., and any range therein, including the endpoints.

Bottom stream 207 is fed to aquaprocessing unit 211 and processed under conditions to produce diesel 212, fuel oil 213, and an aquaprocessed top stream 208 which is fed to downstream steam cracker effluent separator 210.

In aspects, other hydrocarbon feeds 114 may be combined with crude 101 prior to entering the crude flash unit or they may be combined with the bottom feed 105 and optionally also the hydrogen 112 prior to entering aquaprocessing unit 106.

Unit 122 could also be a butane dehydrogenation unit or a butane to ethane conversion unit from where ethane is fed to an ethane cracker.

In a steam cracking process, the saturated hydrocarbons are broken down into smaller, often unsaturated, hydrocarbons such as ethylene and propylene by diluting the mixed hydrocarbon feed with steam and heating the mixture in a furnace in the absence of oxygen. The steam cracking reaction can have a residence times of 50-1000 milliseconds. Mixed steam cracking unit can have a fractionation unit (not shown) or a gas fractionation unit (not shown) capable of separating ethane from the olefin product stream. Such fractionation units are well known in the art.

The ethane stream 118 can include other gaseous compounds such as propane and butane. In steam cracking unit 104, gaseous ethane can be diluted with steam and heated to a temperature of 775° C. to 860° C. (e.g., 775° C., 800° C., 825° C., 850° C., 860° C., or any value or range there between) and/or a pressure of 0.2 MPa to 0.3 MPa (e.g., 0.2 MPa, 0.21 MPa, 0.22 MPa, 0.23 MPa, 0.24 MPa, 0.25 MPa, 0.26 MPa, 0.27 MPa, 0.28 MPa, 0.30 MPa, or any value or range there between) in one or more furnaces to produce ethylene and/or C2+hydrocarbons, as well as higher olefins, i.e. C3⁺.

Aspects relate to method for producing olefins and diesel and/or fuel oil, the method comprising the steps of: feeding a hydrocarbon feed into a first separation unit to produce a first stream having a boiling point less than 200° C. and a second stream having a boiling point above 200° C.; feeding the second stream to a first aquaprocessing unit having a first catalyst therein and aquaprocessing the second stream at a temperature of from greater than 400° C. to 500° C. to produce an aquaprocessing effluent containing 95 wt. % hydrocarbons having a boiling point below 350° C.; separating the aquaprocessing effluent to form a first stream comprising the diesel having a boiling range of from 220 to 370° C.; a third stream comprising fuel oil, a liquid stream having a boiling point in the range of from 35 to 400° C., and a first gaseous stream having a boiling point of less than 200° C.; feeding the first gaseous stream and the first stream to a steam cracker; and steam cracking the first gaseous stream and the first stream in the steam cracker to produce olefins. Note that different quantities of diesel and fuel oil depending on severity of aquaprocessing. For example at aquaprocessing reactor temperature of 450° C., fuel oil could be, e.g., 1-2%. The liquid stream may also be fed to the to the steam cracker. A second liquid stream is produced in the first flash unit, and feeding the second liquid stream to the steam cracker, wherein the second liquid stream has a boiling point ranging from 35° C. to 200° C. The hydrocarbon feed comprises crude oil. The aquaprocessing unit comprises a fixed bed reactor. The aquaprocessing catalyst comprises particulate and dissolved catalysts.

Aspects relate to a method for producing olefins and diesel, the method includes the steps of: feeding a hydrocarbon feed into a first separation unit to produce a first stream having a boiling point less than 200° C., and a second stream having a boiling point above 200° C.; feeding the second stream to a first aquaprocessing unit having a first catalyst therein and aquaprocessing the third stream at a temperature of from greater than 400° C. to 500° C. to produce an aquaprocessing effluent having a boiling point of 95% boiling below 350° C.; separating the aquaprocessing effluent to form a first stream comprising diesel having a boiling range of from 220 to 370° C.; a third stream comprising fuel oil, a liquid stream having a boiling point in the range of from 35 to 400° C., and a second gaseous stream having a boiling point of less than 200° C.; combining the second gaseous stream and the first stream to form a combined gas stream;. The liquid stream is fed to the steam cracker. The combined gas stream is fed to a steam cracker; and steam cracking the combined gas stream in the steam cracker to produce olefins. In aspects, a second liquid stream is produced in the flash unit, and feeding the second liquid stream to the steam cracker, wherein the second liquid stream has a boiling point ranging from 35° C. to 200° C. The hydrocarbon feed comprises crude oil. In aspects, the aquaprocessing unit comprises a fixed bed reactor. The aquaprocessing catalyst comprises particulate and dissolved catalysts.

Aspects relate to a method for producing olefins and diesel, the method including the steps of feeding a hydrocarbon feed into a first separation unit to produce a first stream having a boiling point less than 200° C., and a second stream having a boiling point above 200° C.; feeding the second stream to a first aquaprocessing unit having a first catalyst therein and aquaprocessing the third stream at a temperature of from greater than 400° C. to 500° C. to produce an aquaprocessing effluent having a boiling point of 95% boiling below 350° C.; separating the aquaprocessing effluent to form a first stream comprising diesel having a boiling range of from 220 to 370° C.; a third stream comprising fuel oil, a liquid stream having a boiling point in the range of from 35 to 400° C., and a third gaseous stream having a boiling point of less than 200° C.; combining the third gaseous stream and the first stream to form a combined gas stream; feeding the combined gas stream to a second distillation unit to produce a distilled product; separating the distilled product into a stream comprising ethane, a stream comprising butane and a stream comprising propane; feeding the steam comprising ethane to a steam cracker; and steam cracking the stream comprising ethane in the steam cracker to produce olefins. The stream comprising propane to a propane dehydrogenation unit or a propane steam cracker. Aspects include feeding the stream comprising butane to a butane dehydrogenation unit or a butane steam cracker to produce olefins or feeding butane to a butane to ethane conversion unit and then feeding ethane produced to an ethane steam cracker to produce olefins.

Hydrocarbon may comprises one or more of the following: crude oil, plastics, oligomers from plastic pyrolysis, synthetic crude oil, crude oil cut, and hydrocarbons from plastics pyrolysis, bio oils.

The steam cracking unit is operated such that a mass ratio of propylene to ethylene (P/E) is 0.45 to 0.68 and preferably 0.6 to 0.68.

Aspects relate to method for processing a hydrocarbon, the method comprising the steps of: feeding a hydrocarbon feed into an optional first separation unit to produce a first stream having a boiling point less than 200° C. and a second stream having a boiling point above 200° C.; feeding the first stream to a first steam cracker; feeding the second stream or hydrocarbon feed to a first fluid catalytic cracker having a first catalyst therein and cracking the second stream in the first fluid catalytic cracker by contacting the second stream with the first catalyst at a temperature of from 450° C. to 730° C. to produce a first liquid effluent having a boiling point ranging from 35 to 550° C. and a gaseous effluent stream containing C1 to C4 hydrocarbons; feeding the first liquid stream to a first aquaprocessing unit having a first catalyst therein and aquaprocessing the first liquid stream at a temperature of from 330 to 500° C. to produce an aquaprocessing effluent having a boiling point ranging up to 400° C.; separating the aquaprocessing effluent to form a second liquid stream having a boiling point range of from 200 to 400° C., and a gaseous stream having a boiling point of less than 200° C.; feeding the gaseous stream and the second liquid stream to the first steam cracker; and steam cracking the gas stream and the second liquid stream in the steam cracker to produce olefins. The overall P/E ratio from the hydrocarbon feed being preferably greater than 0.55.

Non-limiting embodiments of the invention are now described. Embodiment 1 is a method for producing olefins and diesel and/or fuel oil. The method includes the steps of feeding a hydrocarbon feed into a first separation unit to produce a first stream having a boiling point less than 200° C. and a second stream having a boiling point above 200° C.; feeding the second stream to a first aquaprocessing unit having an aquaprocessing catalyst therein and aquaprocessing the second stream at a temperature of from greater than 400° C. to 500° C. to produce an aquaprocessing effluent containing 95% weight of hydrocarbons having a 95% boiling point below 350° C., separating the aquaprocessing effluent to form a third stream containing diesel having a boiling range of from 220 to 370° C.; a fourth stream containing fuel oil, a liquid stream having a boiling point in the range of from 35 to 400° C., and a first gaseous stream having a boiling point of less than 200° C.; feeding the first gaseous stream and the first stream to a steam cracker; and steam cracking the first gaseous stream and the first stream in the steam cracker under conditions to sufficient produce olefins. Embodiment 2 is the method of embodiment 1, further including the step of feeding the liquid stream to the steam cracker. Embodiment 3 is the method of claim 1, wherein a second liquid stream is produced in the first flash unit, and feeding the second liquid stream to the steam cracker, wherein the second liquid stream has a boiling point ranging from 35° C. to 200° C. Embodiment 4 is the method of embodiment 1, wherein the hydrocarbon feed contains crude oil. Embodiment 5 is the method of embodiment 1, wherein the first aquaprocessing unit includes a fixed bed reactor. Embodiment 6 is the method of claim 1, wherein the aquaprocessing catalyst containing a dispersed catalyst and a dissolved catalyst. Embodiment 7 is the method of claim 6, wherein the dissolved catalyst contains an organometallic compound having one or more of Ni, Mo, Co, W, Zr, preferably in the form of metal naphthenates and/or octanoates, and/or wherein the dispersed portion of the catalyst is a selection from the list consisting of: an alkali metal hydroxide or oxide, Ni—Mo oxides, Ni—Mo sulphides, Co—Mo oxides or Co—Mo sulphides, W—Mo oxides, W—Mo sulphides, preferably on alumina or zeolites, or any combination of these.

Embodiment 8 is method for producing olefins and diesel. The method includes the steps of feeding a hydrocarbon feed into a first separation unit to produce a first stream having a boiling point less than 200° C., and a second stream having a boiling point above 200° C.; feeding the second stream to a first aquaprocessing unit having a first catalyst therein and aquaprocessing the second stream at a temperature of from greater than 400° C. to 500° C. to produce an aquaprocessing effluent having a 95% boiling point below 350° C.; separating the aquaprocessing effluent to form a first stream containing diesel having a boiling range of from 220 to 370° C.; a third stream containing fuel oil, a liquid stream having a boiling point in the range of from 35 to 400° C., and a second gaseous stream having a boiling point of less than 200° C.; combining the second gaseous stream and the first stream to form a combined gas stream; feeding the combined gas stream to a steam cracker; and steam cracking the combined gas stream in the steam cracker to produce olefins. Embodiment 9 is the method of embodiment 8, further including the step of feeding the liquid stream and fuel oil to the steam cracker. Embodiment 10 is the method of embodiment 8, wherein a second liquid stream is produced in the flash unit, and feeding the second liquid stream to the steam cracker, wherein the second liquid stream has a boiling point ranging from 35° C. to 200° C. Embodiment 11 is the method of embodiment 8, wherein the hydrocarbon feed contains crude oil. Embodiment 12 is the method of embodiment 8, wherein the first aquaprocessing unit contains a fixed bed reactor. Embodiment 13 is the method of embodiment 8, wherein the first catalyst contains particulate and dissolved catalysts. Embodiment 14 is the method of embodiment 11, wherein the dissolved catalyst includes an organometallic compound having one or more of Ni, Mo, Co, W, Zr, preferably in the form of metal naphthenates and/or octanoates, and/or wherein the dispersed portion of the catalyst is a selection from the list consisting of: an alkali metal hydroxide or oxide, Ni—Mo oxides, Ni—Mo sulphides, Co—Mo oxides or Co—Mo sulphides, W—Mo oxides, W—Mo sulphides, preferably on alumina or zeolites, or any combination of these.

Embodiment 15 is a method for producing olefins and diesel, the method including the steps of feeding a hydrocarbon feed into a first separation unit to produce a first stream having a boiling point less than 200° C., and a second stream having a boiling point above 200° C.; feeding the second stream to a first aquaprocessing unit having a first catalyst therein and aquaprocessing the second stream at a temperature of from greater than 400° C. to 500° C. to produce an aquaprocessing effluent having a 95% boiling point below 350° C.; separating the aquaprocessing effluent to form a first stream containing diesel having a boiling range of from 220 to 370° C.; a third stream containing fuel oil, a liquid stream having a boiling point in the range of from 35 to 400° C., and a third gaseous stream having a boiling point of less than 200° C.; combining the third gaseous stream and the first stream to form a combined gas stream; feeding the combined gas stream to a second flash unit to produce a flashed product; separating the flashed product into a stream containing ethane, a stream containing butane and a stream containing propane; feeding the steam containing ethane to a steam cracker; and steam cracking the stream containing ethane in the steam cracker to produce olefins. Embodiment 16 is the method of embodiment 15, further including the step of feeding the stream containing propane to a propane dehydrogenation unit or a propane steam cracker. Embodiment 17 is the method of claim 15, further including the step of feeding the stream containing butane to a butane dehydrogenation unit or a butane steam cracker.

Embodiment 18 is the method of any of the preceding embodiments, wherein the hydrocarbon feed includes one or more of the following: crude oil, plastics, oligomers from plastic pyrolysis, synthetic crude oil, crude oil cut, hydrocarbons from plastics pyrolysis and bio oil. Embodiment 19 is the method of any of the preceding embodiments wherein the steam cracking unit is operated such that a mass ratio of propylene to ethylene (P/E) is 0.6 to 0.68.

Embodiment 20 is a method for processing a hydrocarbon, the method including the steps of feeding a hydrocarbon feed into an optional first flash unit to produce a first stream having a boiling point less than 200° C. and a second stream having a boiling point above 200° C.; feeding the first stream to a first steam cracker; feeding the second stream or hydrocarbon feed to a first fluid catalytic cracker having a first catalyst therein and cracking the second stream or hydrocarbon feed in the first fluid catalytic cracker by contacting the second stream with the first catalyst at a temperature of from 450° C. to 730° C. to produce a first liquid effluent having a boiling point ranging from 35 to 550° C. and a gaseous effluent stream containing C1 to C4 hydrocarbons; feeding the first liquid stream to a first aquaprocessing unit having a first catalyst containing particulate and dissolved catalysts. therein and aquaprocessing the first liquid stream at a temperature of from 330 to 500° C. to produce an aquaprocessing effluent having a boiling point ranging from up to 400° C.; separating the aquaprocessing effluent to form a second liquid stream having a boiling point range of from 200 to 400° C., and a gaseous stream having a boiling point of less than 200° C.; feeding the gaseous stream and the second liquid stream to the first steam cracker; and steam cracking the gas stream and the second liquid stream in the steam cracker to produce olefins, preferably wherein overall the products have a P/E greater than 0.55.

Embodiment 21 is the method of any of embodiments 1 to 20, wherein the products have a P/E ratio of 0.55. Embodiment 22 is the method of embodiments 20, wherein the steam cracker is operated at a P/E mass ratio of 0.45 to 0.68. Embodiment 23 is the method of any of the proceeding claims, wherein the aqua processing unit is a fixed bed unit.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A method for producing olefins and diesel and/or fuel oil, the method comprising the steps of:
feeding a hydrocarbon feed into a first separation unit to produce a first stream having a boiling point less than 200° C. and a second stream having a boiling point above 200° C.;
feeding the second stream to a first aquaprocessing unit having an aquaprocessing catalyst therein and aquaprocessing the second stream at a temperature of from greater than 400° C. to 500° C. to produce an aquaprocessing effluent comprising 95% weight of hydrocarbons having a 95% boiling point below 350° C.
separating the aquaprocessing effluent to form a third stream comprising diesel having a boiling range of from 220 to 370° C.; a fourth stream comprising fuel oil, a liquid stream having a boiling point in the range of from 35 to 400° C., and a first gaseous stream having a boiling point of less than 200° C.;
feeding the first gaseous stream and the first stream to a steam cracker; and
steam cracking the first gaseous stream and the first stream in the steam cracker under conditions to sufficient produce olefins.

2. The method of claim 1, further comprising the step of:
feeding the liquid stream to the steam cracker.

3. The method of claim 1, wherein a second liquid stream is produced in the first flash unit, and feeding the second liquid stream to the steam cracker, wherein the second liquid stream has a boiling point ranging from 35° C. to 200° C.

4. The method of claim 1, wherein the hydrocarbon feed comprises crude oil.

5. The method of claim 1, wherein the first aquaprocessing unit comprises a fixed bed reactor.

6. The method of claim 1, wherein the aquaprocessing catalyst comprises a dispersed catalyst and a dissolved catalyst, preferably
wherein the dissolved catalyst comprises an organometallic compound having one or more of Ni, Mo, Co, W, Zr, preferably in the form of metal naphthenates and/or octanoates, and/or wherein the dispersed portion of the catalyst is a selection from the list consisting of: an alkali metal hydroxide or oxide, Ni—Mo oxides, Ni—Mo sulphides, Co—Mo oxides or Co—Mo sulphides, W—Mo oxides, W—Mo sulphides, preferably on alumina or zeolites, or any combination of these.

7. A method for producing olefins and diesel, the method comprising the steps of:
feeding a hydrocarbon feed into a first separation unit to produce a first stream having a boiling point less than 200° C., and a second stream having a boiling point above 200° C.;
feeding the second stream to a first aquaprocessing unit having a first catalyst therein and aquaprocessing the second stream at a temperature of from greater than 400° C. to 500° C. to produce an aquaprocessing effluent having a 95% boiling point below 350° C.;
separating the aquaprocessing effluent to form a first stream comprising diesel having a boiling range of from 220 to 370° C.; a third stream comprising fuel oil, a liquid stream having a boiling point in the range of from 35 to 400° C., and a second gaseous stream having a boiling point of less than 200° C.;
combining the second gaseous stream and the first stream to form a combined gas stream;
feeding the combined gas stream to a steam cracker; and
steam cracking the combined gas stream in the steam cracker to produce olefins.

8. The method of claim 7, further comprising the step of feeding the liquid stream and fuel oil to the steam cracker, and/or
wherein a second liquid stream is produced in the flash unit, and feeding the second liquid stream to the steam cracker, wherein the second liquid stream has a boiling point ranging from 35° C. to 200° C.

9. The method of claim 7, wherein the hydrocarbon feed comprises crude oil, and/or
wherein the first aquaprocessing unit comprises a fixed bed reactor.

10. The method of claim 7, wherein the first catalyst comprises particulate and dissolved catalysts, preferably.
wherein the dissolved catalyst comprises an organometallic compound having one or more of Ni, Mo, Co, W, Zr, preferably in the form of metal naphthenates and/or octanoates, and/or wherein the dispersed portion of the catalyst is a selection from the list consisting of: an alkali metal hydroxide or oxide, Ni—Mo oxides, Ni—Mo sulphides, Co—Mo oxides or Co—Mo sulphides, W—Mo oxides, W—Mo sulphides, preferably on alumina or zeolites, or any combination of these.

11. A method for producing olefins and diesel, the method comprising the steps of:
feeding a hydrocarbon feed into a first separation unit to produce a first stream having a boiling point less than 200° C., and a second stream having a boiling point above 200° C.;
feeding the second stream to a first aquaprocessing unit having a first catalyst therein and aquaprocessing the second stream at a temperature of from greater than 400° C. to 500° C. to produce an aquaprocessing effluent having a 95% boiling point below 350° C.;
separating the aquaprocessing effluent to form a first stream comprising diesel having a boiling range of from 220 to 370° C.; a third stream comprising fuel oil, a liquid stream having a boiling point in the range of from 35 to 400° C., and a third gaseous stream having a boiling point of less than 200° C.;
combining the third gaseous stream and the first stream to form a combined gas stream;
feeding the combined gas stream to a second flash unit to produce a flashed product;
separating the flashed product into a stream comprising ethane, a stream comprising butane and a stream comprising propane;
feeding the steam comprising ethane to a steam cracker; and
steam cracking the stream comprising ethane in the steam cracker to produce olefins.

12. The method of claim 11, further comprising feeding the stream comprising propane to a propane dehydrogenation unit or a propane steam cracker, and/or
further comprising feeding the stream comprising butane to a butane dehydrogenation unit or a butane steam cracker.

13. The method of claim 1, wherein the hydrocarbon feed comprises one or more of the following: crude oil, plastics, oligomers from plastic pyrolysis, synthetic crude oil, crude oil cut, hydrocarbons from plastics pyrolysis and bio oil.

14. The method of claim 1, wherein the steam cracking unit is operated such that a mass ratio of propylene to ethylene (P/E) is 0.6 to 0.68.

15. A method for processing a hydrocarbon, the method comprising the steps of:
feeding a hydrocarbon feed into an optional first flash unit to produce a first stream having a boiling point less than 200° C. and a second stream having a boiling point above 200° C.;
feeding the first stream to a first steam cracker;
feeding the second stream or hydrocarbon feed to a first fluid catalytic cracker having a first catalyst therein and cracking the second stream or hydrocarbon feed in the first fluid catalytic cracker by contacting the second stream with the first catalyst at a temperature of from 450° C. to 730° C. to produce a first liquid effluent having a boiling point ranging from 35 to 550° C. and a gaseous effluent stream comprising C1 to C4 hydrocarbons;
feeding the first liquid stream to a first aquaprocessing unit having a first catalyst comprising particulate and dissolved catalysts. therein and aquaprocessing the first liquid stream at a temperature of from 330 to 500° C. to produce an aquaprocessing effluent having a boiling point ranging from up to 400° C.;
separating the aquaprocessing effluent to form a second liquid stream having a boiling point range of from 200 to 400° C., and a gaseous stream having a boiling point of less than 200° C.;
feeding the gaseous stream and the second liquid stream to the first steam cracker; and
steam cracking the gas stream and the second liquid stream in the steam cracker to produce olefins overall products having P/E greater than 0.55.

16. The method of claim 1, wherein the products have a P/E ratio of 0.55.

17. The method of claim 16, wherein the steam cracker is operated at a P/E mass ratio of 0.45 to 0.68.

18. The method of claim 1, wherein the aqua processing unit is a fixed bed unit.
